(19) United States
(12) Patent Application Publication
(10) Pub. No.: US 2018/0332969 A1
(43) Pub. Date: Nov. 22, 2018

(54) MULTI-DIMENSIONAL ROTATION AND TRANSLATION APPARATUS

(71) Applicants: Robert Terzini, Corinth, TX (US); Joshua L Flowers, Silverthorne, CO (US)

(72) Inventors: Robert Terzini, Corinth, TX (US); Joshua L Flowers, Silverthorne, CO (US)

(21) Appl. No.: 15/978,588

(22) Filed: May 14, 2018

Related U.S. Application Data
(60) Provisional application No. 62/506,736, filed on May 16, 2017.

(51) Int. Cl. A47C 3/025; A47C 7/50; A61B 5/00; A61B 3/113; A61B 3/14; A61H 1/00; A61B 3/00

(52) U.S. Cl. CPC A47C 3/0255; A47C 7/506; A47C 3/0257; A61B 5/4023; A61B 5/702; A61H 2201/164; A61B 3/14; A61H 1/001; A61B 3/0008; A61H 2203/0431; A61B 3/113

(57) ABSTRACT

A multi-dimensional rotation and translation apparatus includes a base frame, a yaw frame mounted on the base frame to rotate about a yaw axis, a pitch frame mounted on the yaw frame to rotate about a pitch axis, a roll frame mounted on the pitch frame to rotate about a roll axis, and an occupant support slidably mounted on the roll frame. The base frame may be mounted to translate along a translation frame. The yaw, pitch, and roll axes intersect at a selected location relative to an occupant of the occupant support. A yaw motor is engaged between the base and yaw frames; a pitch motor is engaged between the yaw and pitch frames; and a roll motor is engaged between the pitch and roll frames. The motors are selectively activated to cause relative rotation of the respective frames.

### CROSS REFERENCE TO RELATED APPLICATION

This application claims the benefit of U.S. Prov. App. Ser. No. 62/506,736, filed May 16, 2017, which is incorporated by reference herein.

### BACKGROUND OF THE INVENTION

The present invention is broadly concerned with improvements in apparatus and devices for pivoting and rotating a human subject about multiple axes, in selected sequences and at selected speeds, for diagnosis, treatment, testing, and training purposes.

Sense of balance or equilibrioception is an important physiological sense which helps prevent humans and animals from falling, or having a sensation of falling, when standing, sitting, or moving. Balance is the result of a number of body systems working together: the eyes (visual system), ears (vestibular system), and the body's sense of where it is in space (proprioception) ideally need to be intact. The vestibular system, the region of the inner ear where three semicircular canals converge, works with the visual system to keep objects in focus when the head is moving. This is called the vestibulo-ocular reflex (VOR). The balance system works with the visual and skeletal systems (the muscles and joints and their sensors) to maintain orientation or balance. Visual signals sent to the brain about the body's position in relation to its surroundings are processed by the brain and compared to information from the vestibular, visual, and skeletal systems.

The semicircular canals or semicircular ducts are three semicircular, interconnected tubes located in the innermost part of each ear. The three canals are the horizontal, superior, and posterior semicircular canals. The horizontal or lateral canal detects movement of the head around a vertical (yaw) axis or in a transverse plane. This occurs when the head is turned to the left or right. The superior or anterior semicircular canal detects rotations of the head in around the lateral (pitch) axis, or in the sagittal plane. This occurs, for example, when nodding the head. The posterior semicircular canal detects rotation of the head around an anterior-posterior (roll) axis or in the coronal plane. This occurs, for example, when the head is moved toward a shoulder.

Each canal is filled with a fluid called endolymph and contains motion sensors within the fluids, called statoconia. Statoconia (also called otoconia) are granular and often spherical in shape, between 1 and 50 μm (micrometers) in diameter. At the base of each canal, the bony region of the canal is enlarged, opens into the utricle, and has a dilated sac at one end called the osseous ampulla. Within the ampulla is a mound of hair cells and supporting cells called crista ampullaris. These hair cells have many cytoplasmic projections on the apical surface called stereocilia which are embedded in a gelatinous structure called the cupula. As the head rotates the duct moves but the endolymph lags behind owing to inertia. This deflects the cupula and bends the stereocilia within. The bending of these stereocilia alters an electric signal that is transmitted to the brain. Within approximately 10 seconds of achieving constant motion, the endolymph catches up with the movement of the duct and the cupula is no longer affected, stopping the sensation of acceleration. The specific gravity of the cupula is comparable to that of the surrounding endolymph. Consequently, the cupula is not displaced by gravity, unlike the otolithic membranes of the utricle and saccule. As with macular hair cells, hair cells of the crista ampullaris will depolarise when the stereocilia deflect towards the kinocilium. Deflection in the opposite direction results in hyperpolarisation and inhibition. In the horizontal canal, ampullopetal flow (toward the ampulla) is necessary for hair-cell stimulation, whereas ampullofugal flow (away from the ampulla) is necessary in the anterior and posterior canals.

Rotation about the head primarily activates the saccules and utricles. Peripheral activation of the saccule and utricle leads to activation of the vestibular nuclei that projects to multiple areas of the cortex and subcortex of the brain. One of the most researched subjects is activation of the hippocampus region of the brain from vestibular activation. Activation of the hippocampus has been shown to cause neuroplastic changes within its structures, increasing memory and speed of memory processing. Depending upon the speed of rotation and the axis, blood is pushed to the periphery, causing changes in vascularity and blood flow. Rotation about the torso causes a combination of saccule, utricle, and otolithic activation. Activation of these structures has been shown to change activation of autonomic structures. Depending upon the speed of rotation and the axis, blood is pushed to the head causing changes in vascularity and blood flow.

Horizontal translation causes otolithic activation which can be used therapeutically to correct ocular disorders like hyperopia and convergence/divergence disorders. Vertical translation causes otolithic activation as well as gravireceptor activation. Upward translation increases the activation of the otoliths and gravireceptors, causing the body to perceive the increase of gravitational pull. This, in turn, causes activation throughout areas of the cerebellum. Downward translation decreases the activation of gravireceptors. This type of stimulation causes the perception of falling and can be used therapeutically to suppress a startle reflex or the moro reflex and to change one's perception of the center of pressure. When a person is rotated, the normal response is called an optokinetic reflex. This reflex is measurable, recordable, and reproducible. This measurement and analysis may be utilized to study and diagnose vestibular disorders and diseases.

The Epley maneuver or repositioning maneuver is a procedure which may be used to treat benign paroxysmal positional vertigo (BPPV) of the posterior or anterior canals. The maneuver works by causing free floating particles, or otoconia, from the affected semicircular canal to be relocated, using gravity, back into the utricle, where they can no longer stimulate the cupula, therefore relieving the patient of bothersome vertigo. The maneuver is executed by pivoting the patient about a sequence of axes. Exemplary apparatus for performing the Epley maneuver is described in U.S. Pat. No. 6,800,062 and U.S. Pat. No. 7,559,766, the disclosures of which are incorporated herein by reference.

Candidates for pilot and astronaut training must be tested to assess their tolerance to being positioned in various spatial attitudes and being under the effects of rotations about various axes and their capability of performing various duties when in such attitudes or rotations. Additionally, pilots and astronauts often need to be trained to carry out their assigned duties while under the influence of such attitudes and/or rotations.

Thus, there is a need for apparatus for controlled and coordinated pivoting and rotating a patient or subject about various axes.

### SUMMARY OF THE INVENTION

The present invention provides embodiments of an improved apparatus for rotation of an occupant about and along multiple axes for the purposes of diagnosis, treatment, testing, training, and the like.

An embodiment of a multi-dimensional rotation and translation apparatus according to the present invention includes: an occupant support; an interconnected framework having the occupant support connected thereto and capable of pivoting the occupant support about any of three mutually orthogonal axes; pivoting motors engaged with the framework and selectively operable to pivot the occupant support about any of the three axes; and the framework being constructed such that the three mutually orthogonal axes intersect in a selected spatial relationship to the body of an occupant present in the occupant support. In an embodiment of the apparatus, the framework is constructed such that the three mutually orthogonal axes intersect within the body of an occupant present in the occupant support. In another embodiment of the apparatus, the framework is constructed such that the three mutually orthogonal axes intersect within the head of an occupant present in the occupant support.

The apparatus may include an elongated translation frame having the framework engaged therewith to enable linear translation along the translation frame and a translation motor engaged between the translation frame and the framework and selectively operable to cause reversible translation of the framework along the translation frame. The translation frame may be positioned along a horizontal axis to enable horizontal translation of the framework or along a vertical axis to enable vertical translation of the framework.

The apparatus may include an elongated occupant support frame engaged with the framework hand having the occupant support adjustably positioned therealong. The occupant support frame may include a counterweight adjustably positioned therealong and in spaced relation to the occupant support to thereby counterbalance the position of the occupant support with an occupant present. The occupant support frame may also include a gaze bar adjustably positioned along the occupant support frame to enable alignment thereof with eyes of an occupant of the occupant support. The occupant support frame may include a footrest adjustably positioned therealong and in spaced relation to the occupant support to thereby accommodate the size of an occupant of the occupant support.

The gaze bar may have a camera mounted thereon which is capable of being positioned, aimed, and focused on eyes to an occupant of the occupant support to record eye movement during movement of the framework. Visual Stimulation has been shown to cause activation in multiple regions of the brain dependent on the type of stimulation. A removable screen, such as a video display, may be mounted in front of the occupant, as on the gaze bar, to provide a variety of visual stimulations, including gaze fixation, pursuits, saccades (jerky eye movements including prosaccades, antisaccades, and microsaccades) optokinetic images, plaid patterns, alternating color patterns, and dynamic visual acuity.

Color stimulation has shown to cause activation as different cortical areas depending on the color and intensity of the stimulation. The gaze bar may have a light source positioned thereon in spaced relation to an occupant of the occupant support, the light source being controlled to radiate light toward the occupant, having a color or colors selected to cause a selected neural reaction in the occupant.

The apparatus may be provided with an opaque cover removably positioned about a portion of the framework to control viewing of an environment surrounding the apparatus by an occupant of the occupant support during movement of the framework.

An embodiment of the apparatus includes: a base frame; a yaw frame rotatably connected to the base frame to enable rotation about a yaw axis; a yaw motor engaged between the base frame and the yaw frame and operable to rotate the yaw frame about the yaw axis; a pitch frame rotatably connected to the yaw frame to enable rotation about a pitch axis; a pitch motor engaged between the yaw frame and the pitch frame and operable to rotate the pitch frame about the pitch axis; a roll frame rotatably connected to the pitch frame to enable rotation about a roll axis; a roll motor engaged between the pitch frame and the roll frame and operable to rotate the roll frame about the roll axis; an occupant support connected to the roll frame; and the yaw frame, pitch frame, roll frame, and occupant support frame being sized and interconnected in such a manner that the yaw axis, the pitch axis, and the roll axis intersect in a selected spatial relationship to the body of an occupant present in the occupant support.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is a fragmentary perspective view of an embodiment of a multi-dimensional rotation and translation apparatus according to the present invention.

FIG. 2 is a fragmentary perspective view of a somewhat modified embodiment of the apparatus.

FIG. 3 is a view similar to FIG. 2, with a yaw frame of the apparatus rotated about a yaw axis from the orientation shown in FIG. 2.

FIG. 4 is a view similar to FIG. 3 with the yaw frame rotated further about the yaw axis and showing details of engagement of a roll frame with a pitch frame of the apparatus.

FIG. 5 is a view similar to FIG. 1 and shows the roll frame rotated about a roll axis of the apparatus.

FIG. 6 is a view similar to FIG. 6 and shows the pitch frame rotated about a pitch axis of the apparatus.

FIGS. 7A and 7B are diagrammatic side elevational views of the apparatus with the occupant support and pitch frame thereof shown rotated about the pitch axis to a horizontal orientation and illustrating adjustment of a counterweight along the pitch frame to counterbalance the occupant support with an occupant therein.

FIGS. 8A and 8B are diagrammatic side elevational views of the apparatus with the occupant support and pitch frame shown in an upright orientation and illustrating adjustment of the occupant support along the pitch frame.

FIGS. 9A and 9B are views similar to FIGS. 8A and 8B and illustrate adjustment of a head rest or stabilizer of the apparatus along the pitch frame.

FIGS. 10A, 10B, and 10C are views similar to FIGS. 7A and 7B and illustrate adjustment of a gaze bar of the apparatus along the pitch frame.

FIGS. 11A and 11B are fragmentary diagrammatic perspective views showing one or more colored light emitting diodes (LED's) (FIG. 11A) and a computer display panel (FIG. 11B) mounted on the gaze bar of the apparatus.

FIGS. 12A and 12B are views similar to FIGS. 11A and 11B and illustrate a gaze shell mounted on the pitch frame and gaze bar of the apparatus, with a portion of the gaze shell removed in FIG. 12B.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 1 generally designates an embodiment of a multi-dimensional rotation and translation apparatus according to the present invention. The apparatus 1 includes components which cooperate to pivot or rotate an occupant 2 about or along a plurality of axes, including a vertical yaw axis 4, a transverse pitch axis 5, and/or a front-to-back roll axis 6 for purposes of diagnosis and treatment of certain ailments, testing, training, and research.

In general, the illustrated apparatus 1 generally includes a base frame 10, including a circular base frame ring 11 oriented in a vertical plane intersected by the yaw axis 4 and the pitch axis 5. An annular yaw frame or yaw ring 14 is rotatably engaged with the base frame 10 to enable rotation or pivoting of the yaw frame 14 about the yaw axis 4. An elongated pitch frame 16 has opposite ends rotatably engaged with the yaw frame 14 to enable rotation or pivoting of the pitch frame 16 about the pitch axis 5. An elongated roll frame or occupant support frame 18 is rotatably connected to a middle region of the pitch frame 16 to enable rotation or pivoting of the roll frame 18 about the roll axis 6. The roll frame 18 has an occupant support or chair 20 mounted thereon and is adapted to support the occupant 2 and to retain the occupant therein during pivoting and rotation of the frames 14, 16, and 18.

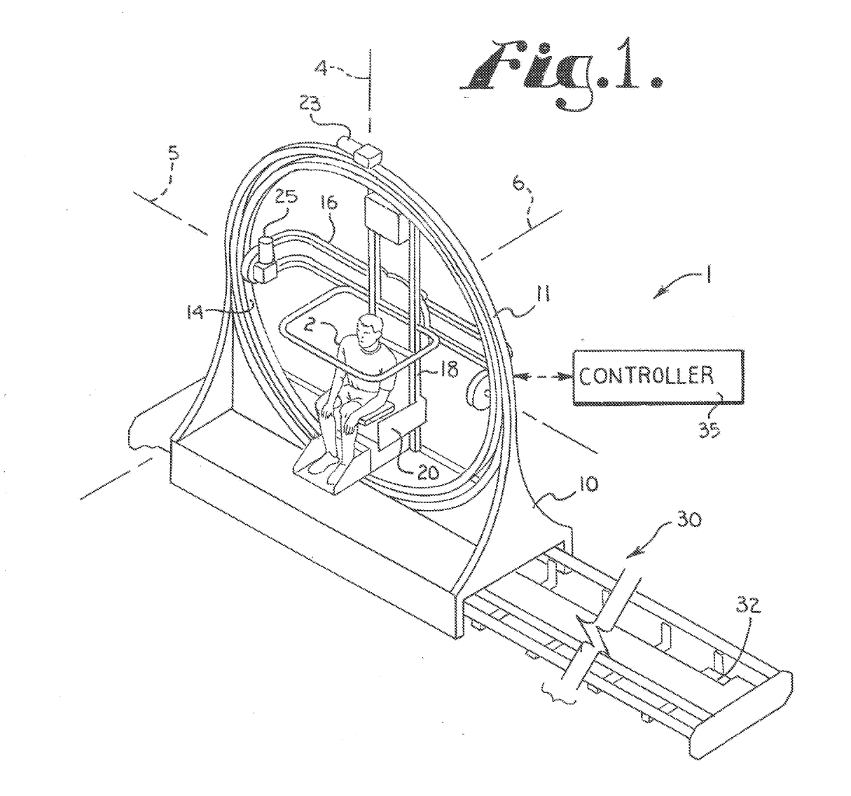

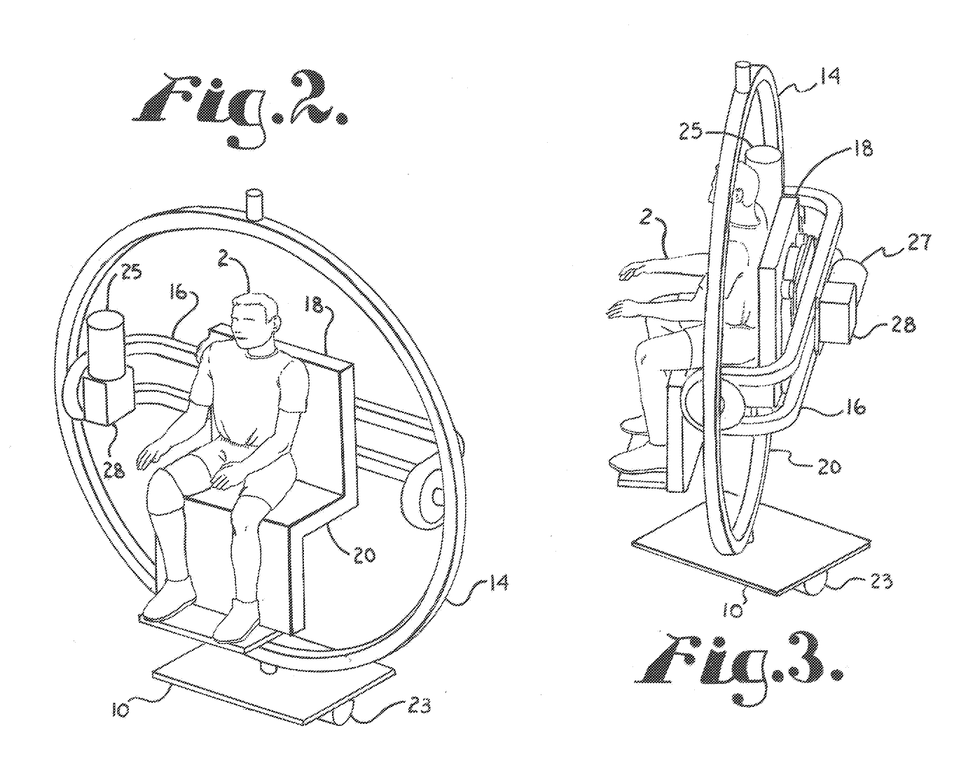

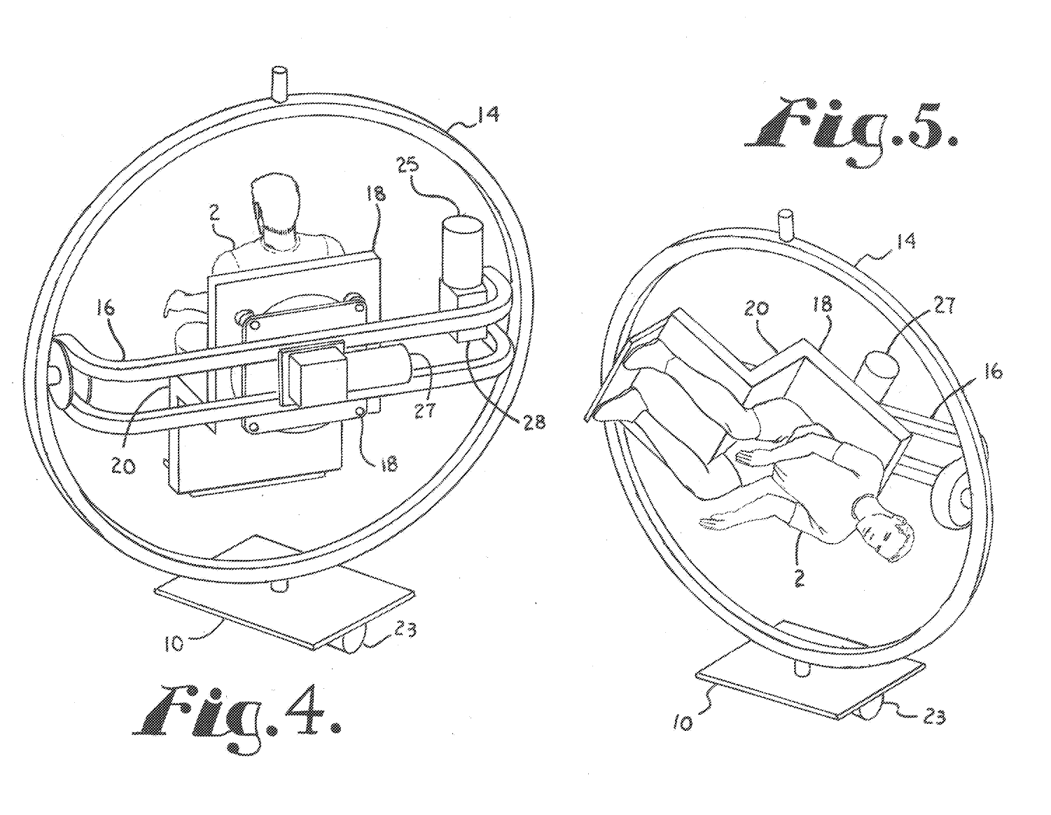

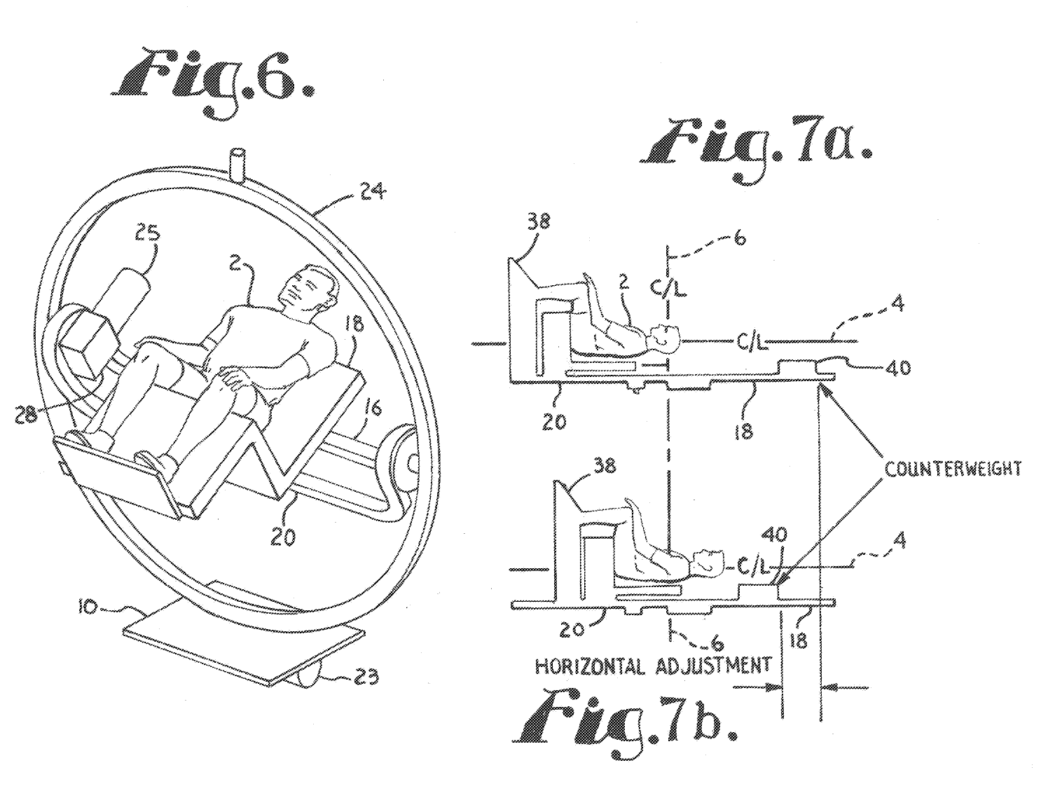

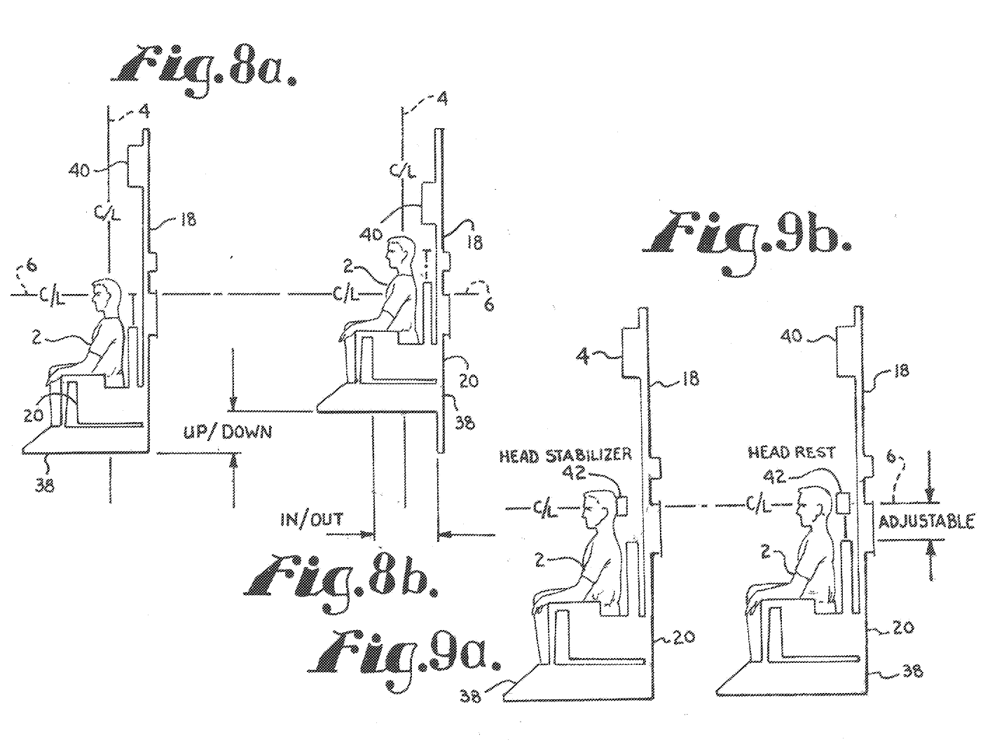

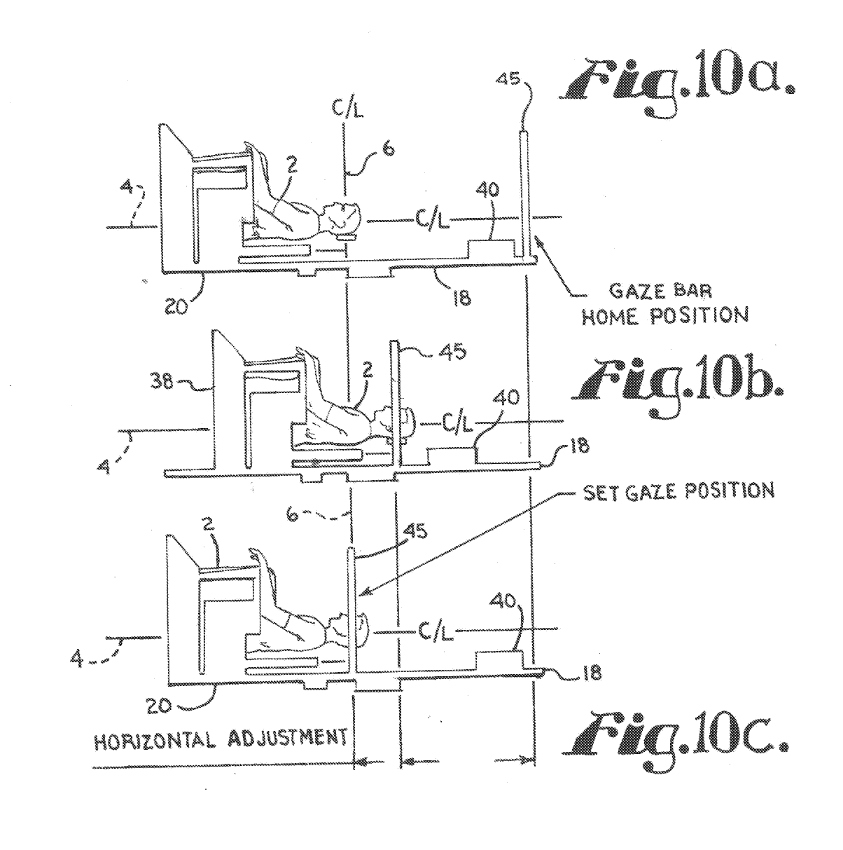

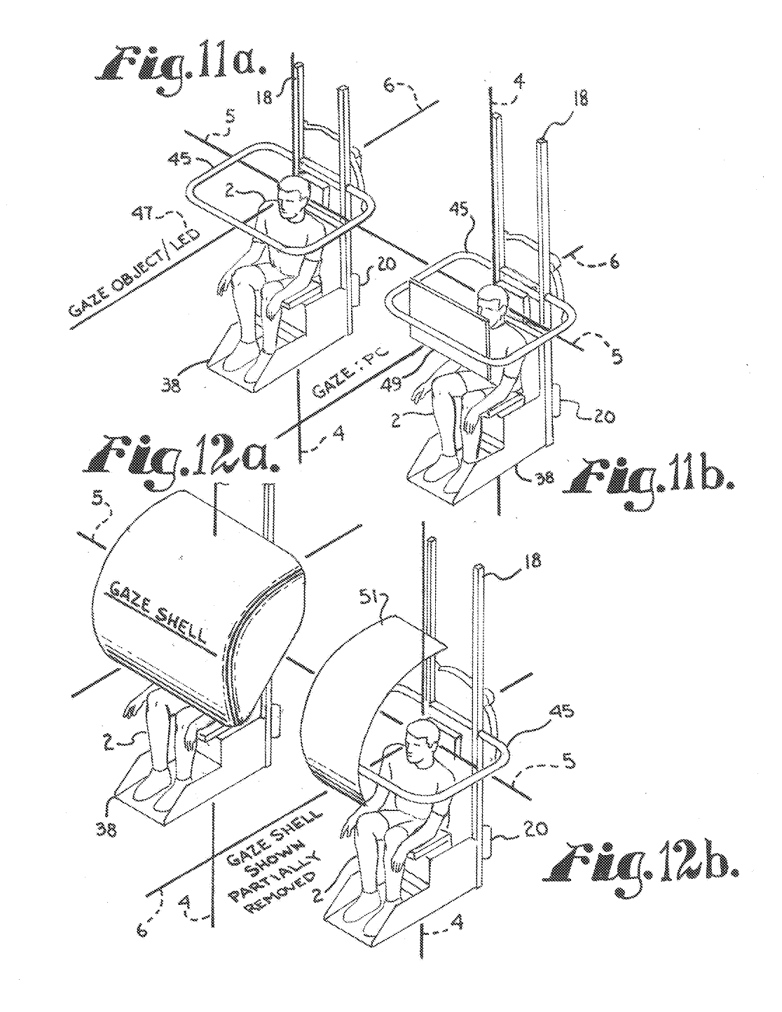

In the illustrated embodiments of the apparatus 1, a yaw motor 23 is engaged between the base frame 10 and the yaw frame 14 and is selectively activated to cause pivoting or rotation of the yaw frame 14 relative to the base frame 10 about the yaw axis 4. The yaw motor 23 may be located at a top of the base frame ring 11, as illustrated in FIG. 1, or at a bottom of the base frame 10, as shown in FIGS. 2-6. A pitch motor 25 is engaged between the yaw frame 14 and the pitch frame 16 and is selectively activated to cause pivoting or rotation of the pitch frame 16 relative to the yaw frame 14 about the pitch axis 5. Although the illustrated pitch motor 25 is shown located to the right of occupant support 20, it is foreseen that it could be located at another location, such as at the left (not shown) of the occupant support 20. A roll motor 27 (FIGS. 3-6) is engaged between the pitch frame 16 and the roll frame 18 and is selectively operated to cause pivoting or rotation of the roll frame 18 relative to the pitch frame 16 about the roll axis 6. The yaw, pitch, and roll motors 23, 25, and 27 may operate through appropriate gear mechanisms 28 (FIGS. 2-4 and 6) to control the torque and speed applied to their respective frames to control the rates of pivoting or rotation thereof.

The illustrated rotation and translation apparatus 1 has the base frame 10 supported on an elongated translation frame 30 to enable reversible translation of the base frame 10 relative to the translation frame 30. The translation frame 30 has a translation motor 32 engaged between the translation frame 30 and the base frame 10 and is selectively activated to cause translation of the base frame 10 on the translation frame 30. The illustrated translation frame 30 is positioned in a horizontal orientation. It is foreseen that the translation frame 30 could, alternatively, be oriented vertically. It is also foreseen that the translation frame 30 could be fixed in or movable to any selected angular orientation (not shown).

The motors 23, 25, 27, and 32 may be interfaced to a control computer or controller 35 which may be programed with sequences of motion of the motors for various effects on the occupant 2. The apparatus may have sensors (not shown) to sense the relative positions of the frames 10, 14, 16, and 18. Such sensors may be interfaced to the controller 35 for use in controlling the motors and other aspects of the apparatus 1.

In the rotation and translation apparatus 1 of the present invention, it is desirable to position the occupant 2 so that pivoting and/or rotation occurs about the yaw, pitch, and/or roll axes 4-6 which have selected relationships to parts of the body of the occupant 2. In the illustrated embodiment of the apparatus 1, the yaw, pitch, and roll axes 4-6 mutually intersect within the head of the occupant 2. It may also be desirable for the axes 4-6 to intersect at another location within the body of the occupant 2, such as the solar plexus, or even external to the body of the occupant 2. Since occupants 2 may have a wide range of body sizes, it is desirable for the location of the occupant support 20 to be adjustable.

Referring to FIGS. 8A and 8B, the occupant support 20 is slidably mounted on the elongated roll frame 18 such a manner that the occupant support 20 can be secured at a desired location along the roll frame 18 in relation to the roll axis 6. It is also desirable that a foot rest 38 be capable of adjustable positioning (not shown) along the roll frame 18 to accommodate the size of the occupant 2 and the position of the occupant support 20 along the roll frame 18.

In order to mechanically balance the assembly of the roll frame 18 with the position of the occupant support 20 with the occupant 2 present, and other devices on the roll frame 18, as will be described, a counterweight 40 (FIGS. 7A and 7B) may be slidably mounted on the roll frame 18 and secured in a position to counterbalance the weight of the occupant support 20, the occupant 2, and any other equipment or devices mounted on the roll frame 18 on the opposite end of the roll frame 18. As illustrated in FIGS. 7A and 7B, position of the counterweight 40 may adjusted with the roll frame 18 pitched to a horizontal orientation. Referring to FIGS. 9A and 9B, the roll frame 18 is preferably provided with a head rest 42 which is mounted thereon in such manner as to enable it to be secured in a selected position along the roll frame 18 to accommodate the size of the occupant 2 and the position of the occupant support 20 along the roll frame 18.

Referring to FIGS. 10A through 11B and 12B, the roll frame 18 may be provided with a visual device support or gaze bar 45 which is slidably mounted on the roll frame 18 to enable it to be secured at selected positions therealong. As shown in FIGS. 10A-10C, the position of the gaze bar 45 may be adjusted with the roll frame 18 pitched to a horizontal orientation. The gaze bar 45 may have a home position at an upper end of the roll frame 18, as shown in FIG. 10A. In operational use, the gaze bar 45 is normally aligned with the eyes of the occupant 2. The gaze bar 45 provides a support for a visual target to be viewed by the occupant 2 during procedures using the apparatus 1. Such visual targets may include light emitting diodes (LED's) 47 (FIG. 11A), a flat panel computer display 49 (FIG. 11B), a video camera (not shown but which may be incorporated into the display 49), or other devices. The LED's 47 may be activated in sequences of colors or brightness levels to determine effects on the occupant 2 before, during, or after movement of the frames 10, 14, 16, or 18. The display 49 may display patterns or sequences of patterns for effects on the occupant during activation of the apparatus 1. A digital camera (not shown) of the display 49 may be used to record characteristic eye movements of the occupant 2 during pivoting or rotation and also facial expressions of the occupant. It is foreseen that parts and components of the apparatus 1 may be painted a neutral color and have lights of selected colors illuminating the apparatus to determine effects thereof on the occupant.

Referring to FIGS. 12A, the apparatus 1 may be provided with a shell or gaze shell 51 which is adjustably positioned on the roll frame 18. The gaze shell 51 moves with the roll frame 18 and restricts the view of the surrounding environment of the occupant 2 during such movement. The gaze shell 51 may be used to avoid distractions to or distress of the occupant 2 during pivoting or rotations, or for other purposes. An inner surface of the gaze shell 51 may be painted a neutral color and have lights of selected colors illuminating the inner surface to determine effects thereof on the occupant 2 during movement thereof.

It is to be understood that while certain forms of the present invention have been described and illustrated herein, it is not to be limited to the specific forms or arrangement of parts described and shown.

What is claimed and desired to be secured by Letters Patent is:

1. A multi-dimensional rotation and translation apparatus comprising:
(a) an occupant support;
(b) an interconnected framework having the occupant support connected thereto and capable of pivoting the occupant support about any of three mutually orthogonal axes;
(c) pivoting motors engaged with the framework and selectively operable to pivot the occupant support about any of the three axes; and
(d) the framework being constructed such that the three mutually orthogonal axes intersect in a selected spatial relationship to the body of an occupant present in the occupant support.

2. An apparatus as set forth in claim 1 wherein:
(a) the framework is constructed such that the three mutually orthogonal axes intersect within the body of an occupant present in the occupant support.

3. An apparatus as set forth in claim 1 wherein:
(a) the framework is constructed such that the three mutually orthogonal axes intersect within the head of an occupant present in the occupant support.

4. An apparatus as set forth in claim 1 and including:
(a) an elongated translation frame having the framework engaged therewith to enable linear translation along the translation frame; and
(b) a translation motor engaged between the translation frame and the framework and selectively operable to cause reversible translation of the framework along the translation frame.

5. An apparatus as set forth in claim 4 wherein:
(a) the translation frame is positioned along a horizontal axis to enable horizontal translation of the framework.

6. An apparatus as set forth in claim 4 wherein:
(a) the translation frame is positioned along a vertical axis to enable vertical translation of the framework.

7. An apparatus as set forth in claim 1 and including:
(a) an elongated occupant support frame engaged with the framework hand having the occupant support adjustably positioned therealong.

8. An apparatus as set forth in claim 7 and including:
(a) a counterweight adjustably positioned along the occupant support frame in spaced relation to the occupant support to thereby counterbalance the position of the occupant support with an occupant present.

9. An apparatus as set forth in claim 7 and including:
(a) a gaze bar adjustably positioned along the occupant support frame to enable alignment thereof with eyes of an occupant of the occupant support.

10. An apparatus as set forth in claim 9 and including:
(a) a camera capable of being positioned, aimed, and focused on eyes to an occupant of the occupant support to record eye movement during movement of the framework.

11. An apparatus as set forth in claim 7 and including:
(a) a footrest adjustably positioned along the occupant support frame in spaced relation to the occupant support to thereby accommodate the size of an occupant of the occupant support.

12. An apparatus as set forth in claim 1 and including:
(a) an opaque cover removably positioned about a portion of the framework to control viewing of an environment surrounding the apparatus by an occupant of the occupant support during movement of the framework.

13. An apparatus as set forth in claim 1 and including:
(a) a light source positioned in spaced relation to an occupant of the occupant support; and
(b) the light source being controlled to radiate light toward the occupant having a color selected to cause a selected neural reaction in the occupant.

14. A multi-dimensional rotation and translation apparatus comprising:
(a) a base frame;
(b) a yaw frame rotatably connected to the base frame to enable rotation about a yaw axis;
(c) a yaw motor engaged between the base frame and the yaw frame and operable to rotate the yaw frame about the yaw axis;
(d) a pitch frame rotatably connected to the yaw frame to enable rotation about a pitch axis;
(e) a pitch motor engaged between the yaw frame and the pitch frame and operable to rotate the pitch frame about the pitch axis;
(f) a roll frame rotatably connected to the pitch frame to enable rotation about a roll axis;
(g) a roll motor engaged between the pitch frame and the roll frame and operable to rotate the roll frame about the roll axis;
(h) an occupant support connected to the roll frame; and
(I) the yaw frame, pitch frame, roll frame, and occupant support frame being sized and interconnected in such a manner that the yaw axis, the pitch axis, and the roll axis intersect in a selected spatial relationship to the body of an occupant present in the occupant support.

15. An apparatus as set forth in claim 14 wherein:
(a) the yaw frame, pitch frame, roll frame, and occupant support frame being sized and interconnected in such a manner that the yaw axis, the pitch axis, and the roll axis intersect within the body of an occupant present in the occupant support.

16. An apparatus as set forth in claim 14 wherein:
(a) the yaw frame, pitch frame, roll frame, and occupant support frame being sized and interconnected in such a manner that the yaw axis, the pitch axis, and the roll axis intersect within the head of an occupant present in the occupant support.

17. An apparatus as set forth in claim 14 wherein:
(a) the roll frame is elongated and has the occupant support adjustably positioned therealong.

18. An apparatus as set forth in claim 17 and including:
(a) a counterweight adjustably positioned along the roll frame in spaced relation to the occupant support to thereby counterbalance the position of the occupant support with an occupant present.

19. An apparatus as set forth in claim 17 and including:
(a) a gaze bar adjustably positioned along the roll frame to enable alignment thereof with eyes of an occupant of the occupant support.

20. An apparatus as set forth in claim 19 and including:
(a) a camera positioned on the gaze bar which is aimed and focused on eyes of an occupant of the occupant support to record eye movement during movement of the occupant support.

21. An apparatus as set forth in claim 19 and including:
(a) a light source positioned on the gaze bar in spaced relation to an occupant of the occupant support; and
(b) the light source being controlled to radiate light toward the occupant having a color selected to cause a selected neural reaction in the occupant.

22. An apparatus as set forth in claim 17 and including:
(a) a footrest adjustably positioned along the roll frame in spaced relation to the occupant support to thereby accommodate the size of an occupant of the occupant support.

23. An apparatus as set forth in claim 14 and including:
(a) an opaque cover removably positioned about at least a portion of the occupant support to control viewing of an environment surrounding the apparatus by an occupant of the occupant support during movement of the occupant support.

24. An apparatus as set forth in claim 14 and including:
(a) an elongated translation frame having the base frame engaged therewith to enable linear translation along the translation frame; and
(b) a translation motor engaged between the translation frame and the base frame and selectively operable to cause reversible translation of the base frame along the translation frame.

25. An apparatus as set forth in claim 24 wherein:
(a) the translation frame is positioned along a horizontal axis to enable horizontal translation of the base frame.

26. An apparatus as set forth in claim 24 wherein:
(a) the translation frame is positioned along a vertical axis to enable vertical translation of the base frame.

27. A multi-dimensional rotation and translation apparatus substantially as described and illustrated.
